(19) United States
(12) Patent Application Publication
(10) Pub. No.: US 2008/0294174 A1
(43) Pub. Date: Nov. 27, 2008

(54) METHODS AND APPARATUS FOR PERICARDIAL ACCESS

(75) Inventors: Earl Bardsley, Newton, MA (US); Jianlu Ma, Maple Grove, MN (US); Jean Paul Rasschaert, Eagan, MN (US)

Correspondence Address: HAMRE, SCHUMANN, MUELLER & LARSON, P.C., P.O. BOX 2902, MINNEAPOLIS, MN, 55402-0902, US

(73) Assignee: Epitek, Inc., Bloomington, MN (US)

(21) Appl. No.: 12/118,915

(22) Filed: May 12, 2008

Related U.S. Application Data
(60) Provisional application No. 60/939,199, filed on May 21, 2007.

(51) Int. Cl. A61M 29/00

(52) U.S. Cl. 606/108; 206/571; 600/434; 606/186

(57) ABSTRACT

An access tube has a central passage and at least one lumen extending axially between the central passage and an outside wall of the access tube. The lumen carries therein a piercing member for accessing an anatomic space, such as a pericardial space between the parietal pericardia and the visceral pericardia. While the access tube is positioned against the parietal pericardium, a distal end of the piercing member engages and penetrates the parietal pericardium. The distal end of the piercing member entering the anatomic space can then allow the piercing member to advance and form into a supporting structure to create a working space. After the working space is created, an access device can be introduced through the access tube into the pericardial space to perform a variety of procedures inside a patient.

### CROSS-REFERENCE TO RELATED APPLICATIONS

The present application claims the benefit of U.S. Provisional Patent Application Ser. No. 60/939,199 entitled “METHODS AND APPARATUS FOR PERICARDIAL ACCESS,” filed on May 21, 2007, which is herewith incorporated by reference in its entirety.

### FIELD

The present disclosure relates generally to medical devices and methods. More particularly, the present disclosure relates to methods and devices for accessing the pericardial space in a minimally invasive manner.

### BACKGROUND

The human heart is enveloped within a tissue structure referred to as the pericardium. The pericardium includes two major portions. The portion of the pericardium which lies immediately over the surface of the heart is referred to as the visceral pericardium. The second portion is formed as a sac around the visceral pericardium and is referred to as the parietal pericardium. Normally, the visceral and parietal pericardia lie in close contact with each other and are separated only by a thin layer of pericardial fluid. The space (really more of a potential space) between the visceral and parietal pericardia is referred to as the pericardial space.

Access to the pericardial space can be necessary or beneficial under a variety of circumstances. Open surgical access can be obtained via open sternotomy where the patient's sternum is divided and the parietal pericardium exposed. Such an approach, however, is highly traumatic, requiring general anesthesia and useful only under compelling circumstances. Access to the pericardial space can also be achieved using a thoracoscopic approach. Under general anesthesia, the left lung is deflated after which multiple holes are made for the thoracoscope and various instruments. The pericardium is then entered using standard videoscopic techniques. The thoracoscopic approach typically requires the placement of a chest tube and admission to the hospital for the initial 1-2 post-operative days. In other approaches, the pericardial space can be approached from a skin incision made below the xiphoid through which the parietal pericardium is identified.

It would be desirable to provide additional and improved methods and apparatus for the minimally invasive access to a patient's pericardial space. The methods and devices should be suitable for a wide variety of minimally invasive approaches to the pericardium, including at least intercostal/transthoracic and subxiphoid approaches, and the like. The methods and devices should further provide for secure and stable capture of the parietal pericardium and permit the opening of a large space or volume between the parietal and visceral pericardia. Such access methods and apparatus should be useful for a wide variety of procedures to be performed in the pericardial space, including fluid withdrawal, drug delivery, diagnostic and therapeutic electrophysiology procedures, pacemaker lead implantation, defibrillator lead placement, transmysocardial revascularization, transmysocardial revascularization with drug delivery, placement of the left ventricular assist devices, placement of the arterial bypass graphs, in situ bypass, i.e., coronary artery-venous fistulae, placement of drug delivery depots, closure of the left arterial appendage, and the like.

U.S. Pat. No. 6,423,051 discusses that an anchor structure of an access tube engages and captures the outer surface of the parietal pericardium and draws the parietal pericardium away from the visceral pericardium to create an enlarged pericardial space. After such enlargement, a needle or other access device can be introduced through the access tube into the pericardial space to provide access for a wide variety of purposes, including aspiration, infusion and guidewire placement.

### SUMMARY

The present disclosure relates to apparatuses, systems, kits and methods for accessing an anatomic space having a wall with an outer surface. In one embodiment, an apparatus is provided for accessing an anatomic space having a wall with an outer surface. The apparatus includes 1) a tube having a central passage and at least one lumen extending axially between the central passage and an outside wall of the apparatus, and 2) at least one piercing member carried by the lumen. The piercing member has a distal end configured to penetrate the wall. Once the wall is penetrated, the pericardium can be pulled away from the heart's surface. After the space is formed, the piercing member can then be advanced to hold that space open. Another method is to pierce the wall and then advance the piercing member to form the space. A distal portion of the piercing member is configured to advance through the lumen and into the anatomic space. The piercing member is configured to form a supporting structure, and expand the anatomic space to create a working space inside the anatomic space without injuring the wall.

In another embodiment, a system is provided for accessing an anatomic space having a wall with an outer surface. The system includes 1) an access tube having a central passage, at least one lumen extending axially between the central passage and an outside wall of the access tube, and at least one piercing member carried by the lumen, and 2) an access device. The piercing member has a distal end configured to penetrate the wall. A distal portion of the piercing member is configured to advance through the lumen and into the anatomic space. The piercing member is configured to form a supporting structure, and expand the anatomic space to create a working space inside the anatomic space without injuring the wall. The access device is configured to pass through the access tube and into the anatomic space, after the working space is created.

In a further embodiment, an access tube and an access device as described may be packaged into a kit for accessing a pericardial space between a visceral pericardium and a parietal pericardium.

In yet another embodiment, a method is provided for accessing an anatomic space having a wall having an outer surface. The method includes a) providing an access tube having a central passage and at least one lumen extending axially between the central passage and an outside wall of the access tube, where the lumen carries therein a piercing member; b) positioning a distal end of the piercing member proximate to the outer surface of the wall; c) penetrating the distal end of the piercing member through the wall; and d) advancing the piercing member through the lumen and into the anatomic space, and e) expanding the anatomic space to create a working space inside the anatomic space without injuring the wall when a distal portion of the piercing member forms into a supporting structure.

### BRIEF DESCRIPTION OF THE DRAWINGS

FIG. 1 is an embodiment of an apparatus for accessing an anatomic space.

FIG. 2 is the apparatus of FIG. 1 with one embodiment of piercing members where a distal portion of each piercing member extending out and forming one embodiment of a supporting structure.

FIG. 3 is an enlarged view of a distal end of the access tube of FIG. 1.

FIG. 4 is an embodiment of a system for accessing an anatomic space.

FIGS. 5A-5B show the apparatus of FIG. 1 and a sheath for introducing the apparatus.

FIGS. 6A-6G show uses of the system of FIG. 4 for accessing the pericardial space.

FIG. 7 is an alternative use of the system of FIG. 4 for accessing a pericardial space.

FIG. 8 is one embodiment of a kit constructed in accordance with the principles of the present disclosure.

FIG. 9 is a schematic of one embodiment of holding a piercing member in a lumen of an access tube.

FIG. 10 is a schematic of another embodiment of holding a piercing member in a lumen of an access tube.

FIG. 11 shows one embodiment of an access device.

### DETAILED DESCRIPTION

The present disclosure relates to apparatus, systems, kits and methods for accessing an anatomic space having a wall with an outer surface and creating a working space to perform a variety of procedures inside the working space. The wall may include a membrane, a capsule or the adventia, muscularis and endothelial layers of a hollow organ or vessel. The apparatuses, systems, kits and methods are particularly useful for minimally invasive access procedures, but could also be used for accessing internal anatomic spaces where initial access to the wall of the outer surfaces is achieved via open surgical or other techniques. The disclosed apparatus can be particularly useful for accessing a patient's pericardial space for performing a wide variety of procedures, generally as set forth above.

The phrase “anatomic space” is meant to include any natural, potential, or created space or cavity within a patient's body where it may be desirable to gain access for surgical, diagnostic, therapeutic, or any other purpose. In some embodiments, the anatomic space is within an organ or structure located beneath the patient's skin, such as the pericardial space which lies between the visceral and parietal pericardia, both of which lie beneath the chest wall and rib cage. Other internal organs which may be accessed include the intestines, fallopian tubes, gall bladder, kidneys, and the like.

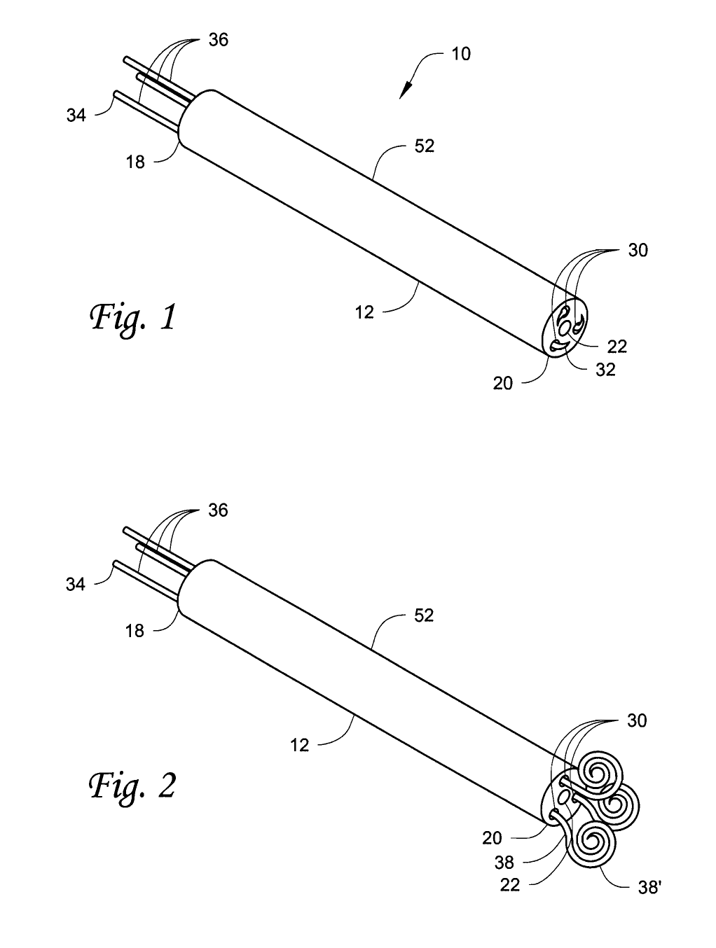

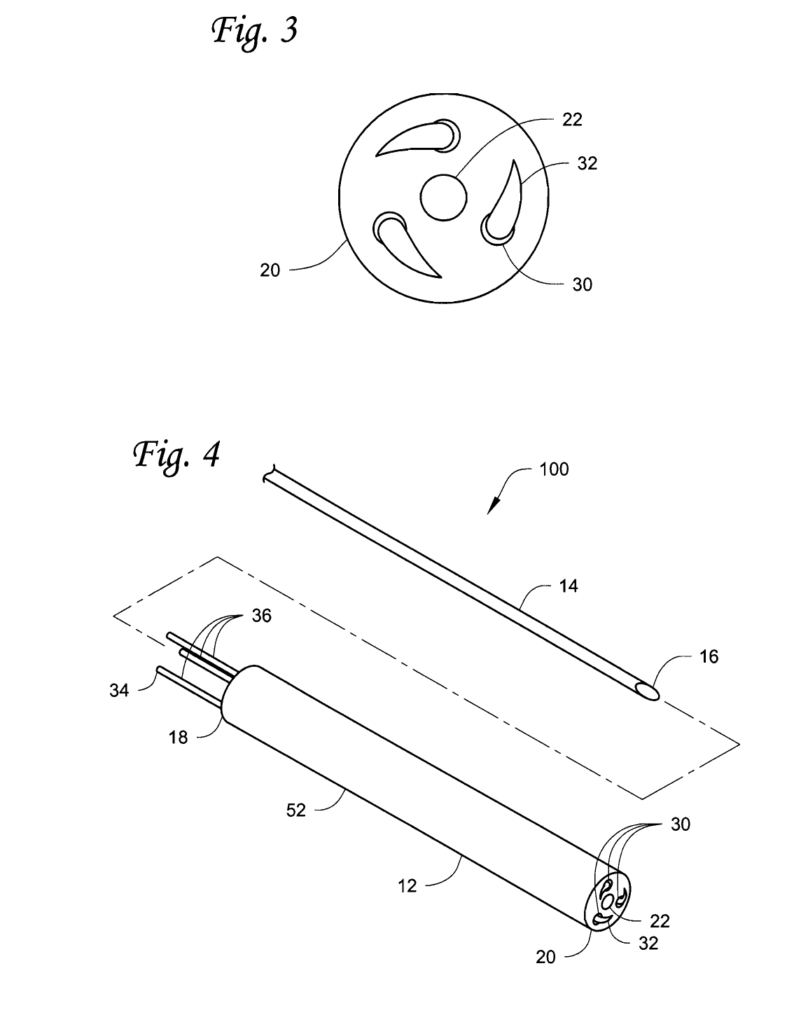

FIGS. 1-4 show an embodiment of an apparatus 10 for accessing an anatomic space. The apparatus 10 includes an access tube 12 having a central passage 22 and a plurality of lumens 30 extending axially between the central passage 22 and an outside wall 52 of the access tube 12. The access tube 12 comprises a tubular or cylindrical body having a proximal end 18 and a distal end 20. The access tube 12 may have a wide variety of specific structures and configurations, but generally is an elongated body, typically having a length in the range from 10 cm to 30 cm, more usually from 18 cm to 24 cm, and a relatively narrow maximum width, in some cases, having a diameter in the range from 3 mm to 20 mm, more usually from 4 mm to 10 mm. The access tube 12 can be made of a variety of materials, for example, Pebax®, polypropylene, polyethylene, Nylon, stainless steel, Nitinol, or the like.

In one embodiment, the central passage 22 is generally configured so that an access device 14 for performing treatment in the anatomic space may easily pass through the access tube 12 (access devices are described in more detail in FIGS. 4, 5A-5G, and FIG. 11). In one embodiment, the central passage 22 generally has the same length as that of the access tube 12, and in some cases has a diameter in the range from 1 mm to 6 mm, more usually from 1.5 mm to 4 mm. The lumens 30 may have a variety of structures, but can generally be configured so that piercing members 36 (described below) may easily advance through the lumens 30. For example, in one embodiment, the lumens 30 can be configured so that the piercing members 36 may slide therein. The lumens 30 generally have the same length as that of the access tube, and in some cases has a diameter in the range from 0.01 inch to 0.04 inch, more usually from 0.02 inch to 0.03 inch. In one embodiment, the access tube may include a plurality of lumens, for example, three lumens as shown in FIGS. 1-4. It will be appreciated that a single lumen, rather than a plurality of lumens, may be formed axially between the central passage 22 and an outside wall of the access tube 12. It will be further appreciated that more than three lumens may be employed.

Each lumen 30 carries a piercing member 36 therein for penetrating the wall of the anatomic space. Each piercing member 36 has a tissue penetrating distal end 32 and a proximal end 34. Each piercing member 36 is configured to engage target tissue on an outer surface of a wall of an anatomic space, and penetrate the wall of the anatomic space that forms or surrounds the anatomic space.

The piercing member 36 can be configured into a variety of structures in a way that a distal portion 38 of the piercing member 36 may form a supporting structure 38′ to expand the anatomic space to create a working space inside the anatomic space. As shown in FIGS. 1, 2 and 4, each of the piercing members 36 is generally elongate, having a tissue-penetrating distal end 32 for passage through the tissue of the wall of the anatomic space. In one embodiment, the tissue-penetrating distal end 32 may be oriented at an angle in the range from 30 degrees to 60 degrees so that it penetrates into tissue as the piercing member 36 is rotated about its central axis.

In one embodiment, the piercing member 36 may be constructed of a length that is substantially as long as or longer than the access tube 12, so that the piercing member 36 may be passed therethrough. The piercing member 36 may also be constructed of a length that is shorter than the access tube 12. In one example, when the piercing member 36 is shorter than the tube 12, another instrument, component, or equipment, or the like can be used to push the piercing member 36 forward so that the piercing member can advance into the anatomic space and can form the supporting structure 38′. It will be appreciated that the piercing member(s) 36 can be extended out of the access tube 12 by the same or different amounts to create a desired working space in the anatomic space. It will be appreciated that an additional instrument, if desired and/or necessary, may be constructed in various ways as one of skill in the art could accomplish, so long as the piercing member would be advanced and function as described herein. It further will be appreciated that the length of the piercing members may vary as desired and/or necessary, as long as the piercing member 36 has a length that is long enough to form a supporting structure 38′ which will be further described herein.

As one example, the piercing member 36 can be made of shape memory materials, such as Nitinol wire. The piercing member can be round Nitinol wire that has been heat shaped into a curled configuration, with a diameter in a range from 2 mm to 20 mm. This curled configuration is pulled back into lumen 30 to straighten the shape. As member 36 is advanced, the member has a natural propensity to return to its curled shape. It will be appreciated that the piercing member 36 may be made of materials other than Nitinol, such as any suitable biasing materials, so long as the piercing member can function to form the supporting structure 38′, which will be further described herein.

When the piercing member 36 is pushed forward to pass through the access tube 12 from the proximal end 18 to the distal end 20 and extends out of the distal end 20 of the access tube 12, a distal portion 38 of the piercing member 36 starts forming the supporting structure 38′. That is, when the piercing member 36 has penetrated the anatomic space and is advanced, it can increase its dimension to form the supporting structure inside the anatomic space. In one example, the supporting structure 38′ is in the form of a coil as shown in FIG. 2, when the piercing member has been advanced and exposed from the distal end 20 of the access tube 12. In yet another embodiment, as the distal portion 38 of the piercing member 36 advances further, the dimension of the supporting structure can continue to increase. It will be appreciated that the surface of the supporting structure 38′ has no edges that would injure the inner surface of the anatomic wall. In some examples, the surface of the supporting structure 38′ can be generally radiused or curved. It will be appreciated that as many piercing members 36 as required to create a working space can be incorporated into the access tube 12. On the other hand, it is also possible that a single piercing member 36 be employed to achieve the above function if desired.

As shown in FIG. 4, an access device 14 is configured to pass through the central passage 22 of the access tube 12 so that it may extend out at the distal end 20 of the access tube 12. In one embodiment, the access device 14 is configured to be introduced through the central passage 22 of the access tube 12 and through a target region of the wall of the anatomic space. When the tissue penetrating distal end 32 of the piercing member 36 has penetrated and captured the tissue and the supporting structure 38′ is formed, the access device 14 can pass through the region of tissue that is being held and stabilized by the piercing members 36. The access device 14 enters into the interior volume of the anatomic space, and can be used to perform a treatment in the working space created by the supporting structure 38′.

The access device 14 may also have a wide variety of specific forms, and can include but is not limited to a needle, stylet, or other elongate structure having a sharpened distal tip for passage through the tissue of the wall of the anatomic space. In one embodiment as shown in FIG. 4, the access device 14 is in the form of a hollow needle or stylet having a sharpened distal end 16.

In some examples, the access device 14 can also have a passage to permit introduction of a guidewire, infusion or aspiration of fluids, placement of leads or other implantable devices, or the like. Placement of a guidewire within the anatomic space may further provide for introduction of a wide variety of other diagnostic and therapeutic catheters and devices. The access device 14 can be longer than the access tube 12 so that it may be passed therethrough. In some cases, the access device 14 has a length in the range from 12 cm to 35 cm, more usually from 20 cm to 26 cm. The maximum width or diameter of the access device 14 may vary, so long as the access device 14 can be introduced through the access tube 12. In some examples, the diameter of the access device 14 may be in the range from 1 mm to 6.0 mm, preferably from 1.5 mm to 4 mm.

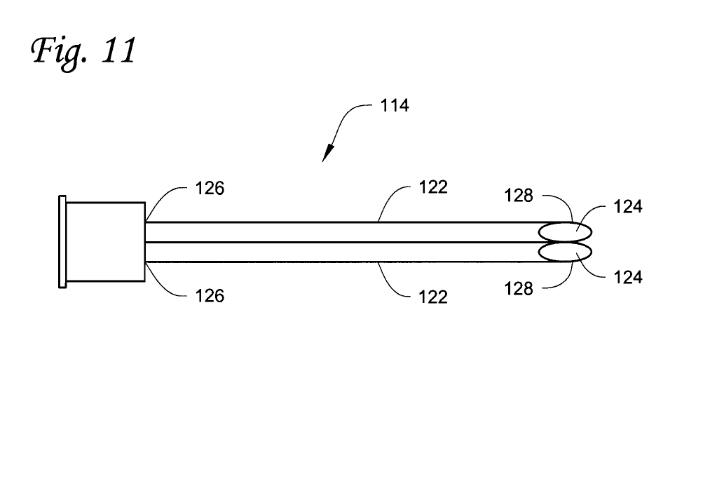

In other embodiments, the access device may be a double-barrel needle. FIG. 11 shows one embodiment of an access device 114 that acts as a double-barrel needle. The access device 114 includes two needles 122 that may be insertable through a central passage of any of the apparatuses described herein. It will be appreciated that the central channel may be configured to be large enough to accommodate insertion of a double-barrel or dual needle type configuration. Each needle includes proximate and distal ends 126, 128 with a channel 124 (entire channel not shown) extending axially therethrough from the proximate end to the sharp edge at the distal end. As shown, the needles 122 are disposed directly adjacent each other. As one example, the channel 124 of one of the needles 122 may be employed for inserting a guidewire (i.e. 40) and/or delivering a contrast media therethrough. The channel 124 of the other needle 122 may be employed for inserting an endoscope (not shown), such as for imaging and visualizing purposes. It will be appreciated that endoscopes are well known and are commercially available, and are not further described. A stop also may be provided at the proximate ends 126 of the needles 122 to prevent over insertion of the needles into a central channel of an apparatus. The stop may also provide for locking capability to the apparatus once the access device 114 is in a desired position.

In operation, the needles 122 access device 114 are inserted into the body, cavity, or lumen of a patient. For example, the access device 114 may inserted via a sub-xiphoid approach or the sternum area for accessing the pericardium. The needles 122 may be advanced to the pericardium, where an endoscope may be inserted through one of the needles 122 to help position the needles 122 just adjacent the pericardium. Once in position, the access device 114 can puncture the pericardium to access, for instance the pericardial space. The needles 122 may then be advanced to introduce contrast media and/or a guidewire.

The double barrel needle stricture helps allow for a coordinated effort to access the pericardium, while seeing that the puncture does not enter beyond the pericardium tissue into the heart tissue. Thus, the double barrel needle structure also can allow an operator to perform multiple procedures while accessing the pericardial space. For instance, one barrel may be employed to house a small diameter endoscope to visualize the pericardium, while the second barrel may be employed to inject contrast media and/or a guidewire.

It also will be appreciated that the access devices shown and discussed are merely exemplary, and that any of the apparatuses described herein may accommodate various access devices through its central channel for use inside a body of a patient and for use in various procedures.

Turning back to FIG. 4, a system 100 for accessing an anatomic space including the access tube 12 and the access device 14 is shown. It will be appreciated that the construction of the system 100 could be varied in a number of ways for a variety of purposes. For example, the tubular structures of the access tube and/or the access device could be non-linear, telescoping, perforated, or having many other configurations. Additional features, such as additional passages, imaging capabilities, pneumostatic valves, and the like, could also be added within the scope of the present disclosure.

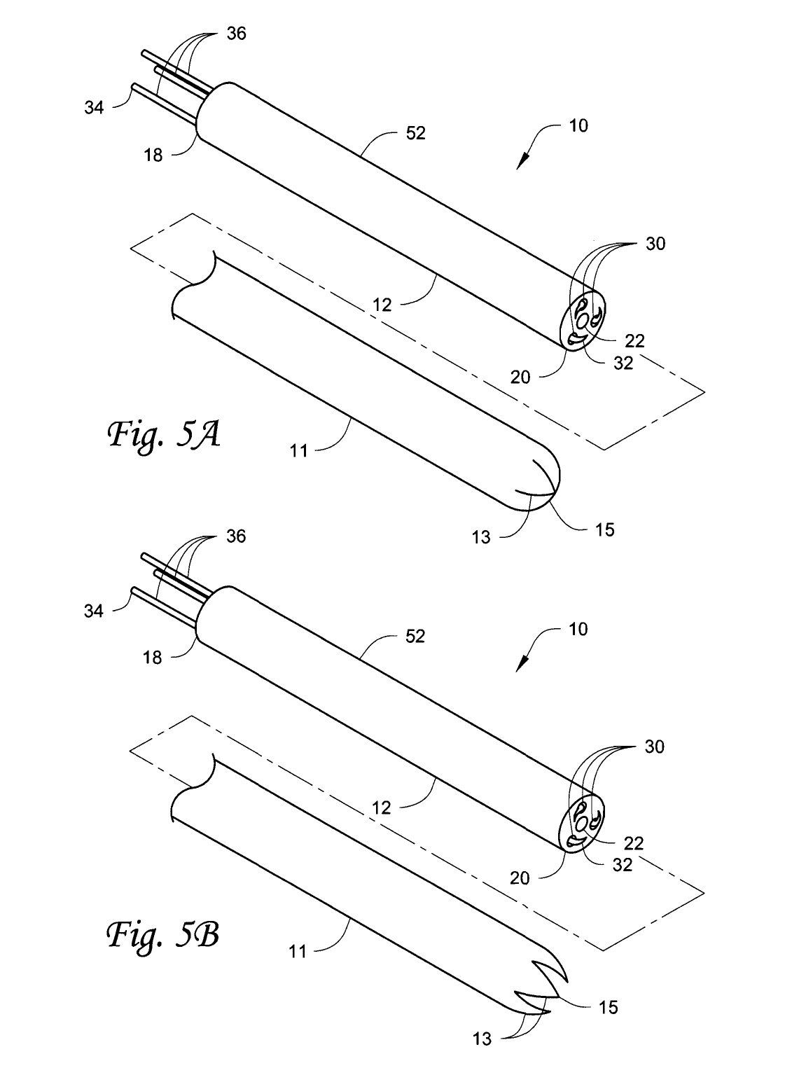

FIGS. 5A, 5B illustrate a sheath 11 for introducing the apparatus 10. The sheath is sized and configured to house the apparatus 10 and includes a plurality of flexible portions, formed on a distal end 15 of the sheath 11. In one embodiment, the flexible portions are flexible petals. In one embodiment, the distal end 15 of the sheath 11 is tapered so that the distal end 15 can be easily placed proximate to the anatomic space. The flexible portions 13 are designed to flex or bend outward to provide an opening through which the apparatus 10 can be exposed from the distal end 15 of sheath 11 at the appropriate time. When the distal end 15 of sheath 11 is placed proximate to the anatomic space, the sheath 11 can be withdrawn. When the sheath 11 is withdrawn, the distal end 20 of the access tube 12 will contact the flexible portions 13. As the sheath 11 is further withdrawn or pulled back, the distal end 20 of the access tube 12 can bend or move the flexible portions 13 outward, so that the distal end 20 of the access tube 12 can be exposed through the opening formed when the flexible portions 13 are moved out of the way. When the apparatus 10 is in position, the sheath 11 can be removed.

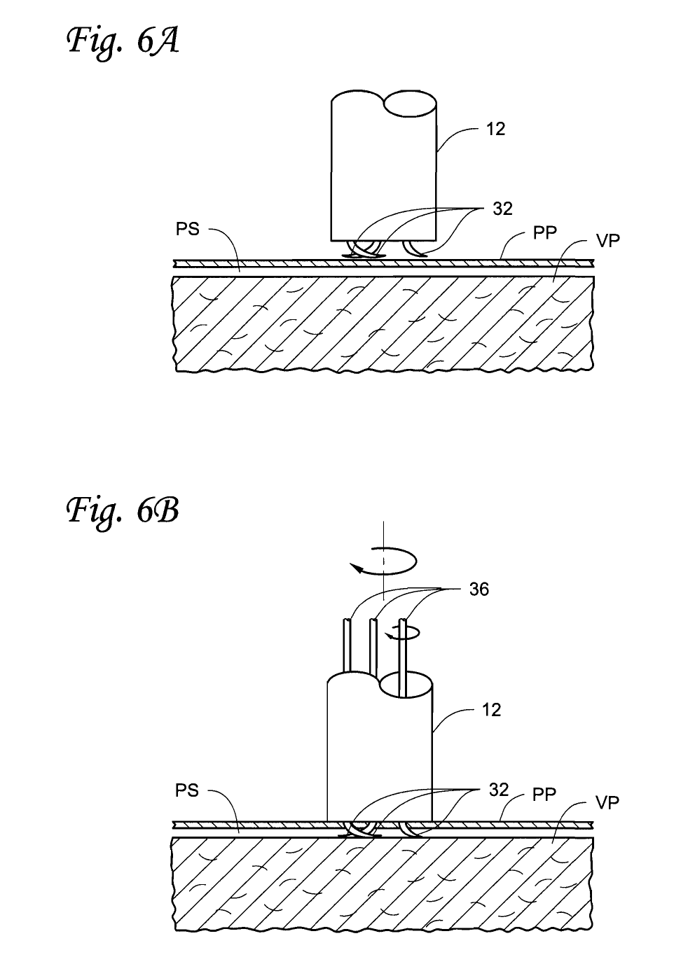

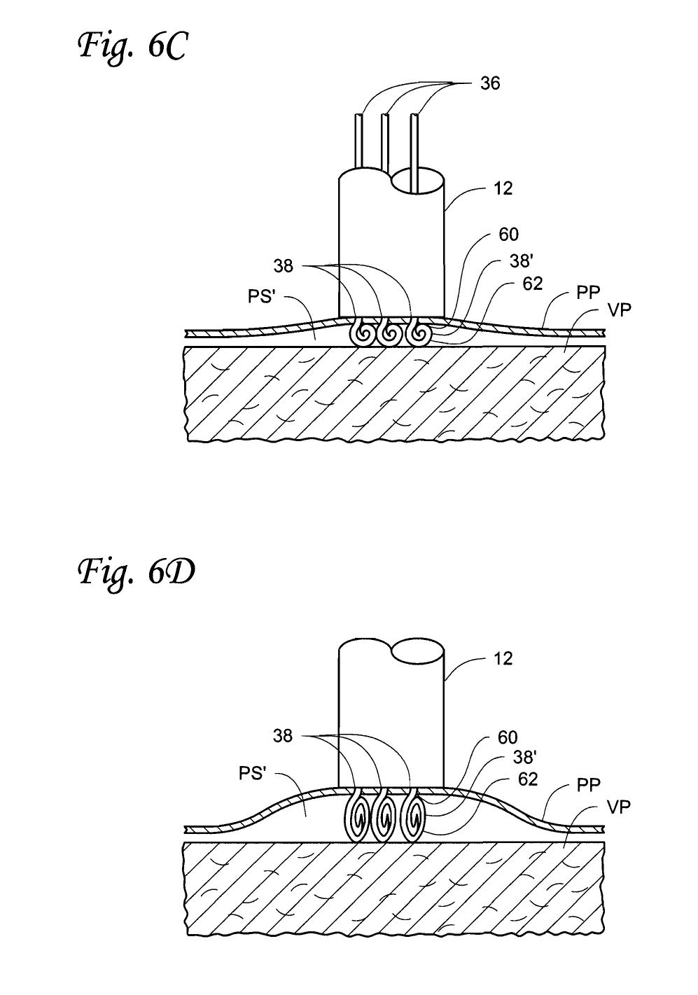

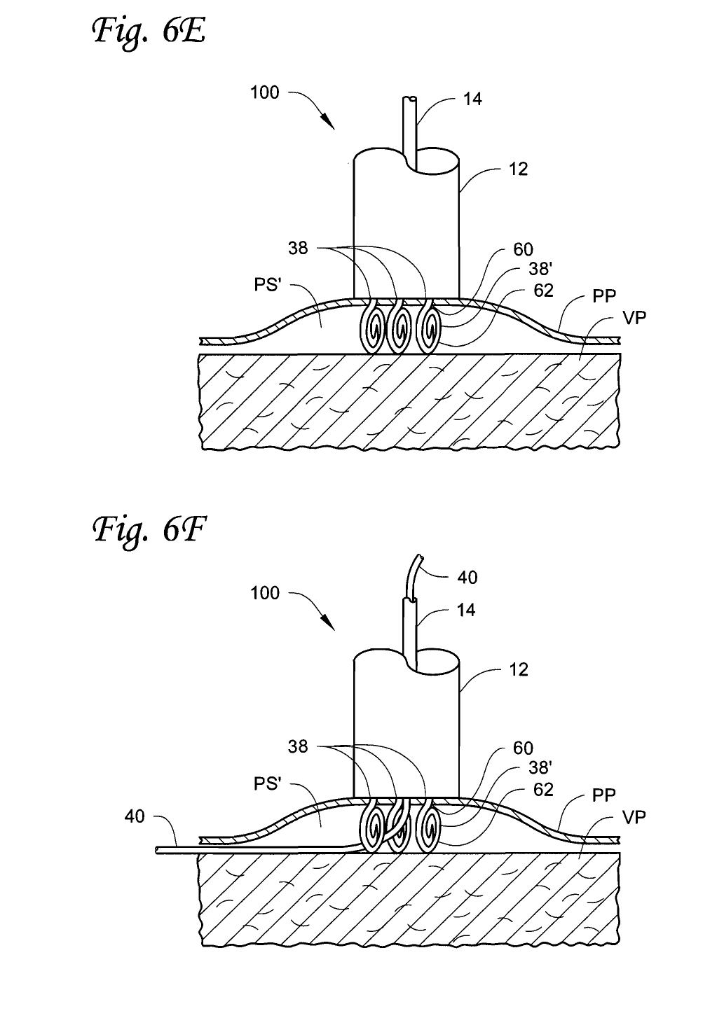

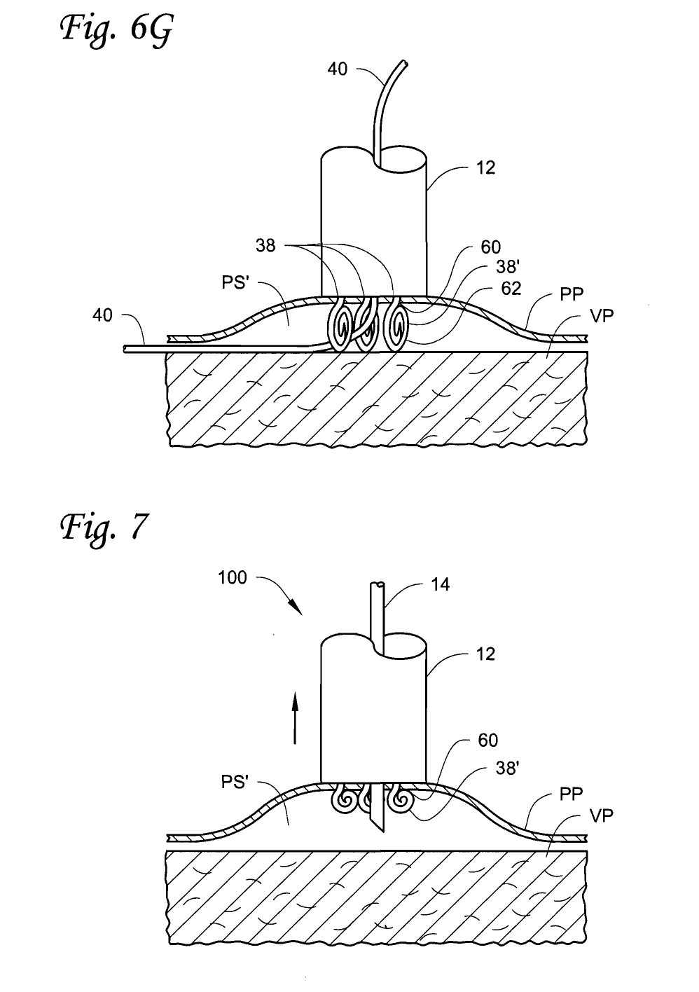

FIGS. 6A-6G, show one embodiment of operation of the system 100, with the anatomic wall being the parietal pericardium PP which overlies the pericardial space PS (or potential space) over a patient's heart. The pericardial space PS′ is formed between the visceral pericardium VP and the parietal pericardium PP, as seen in FIG. 6A. The apparatus 10 can be introduced by a sheath 11 as described in FIGS. 5A, 5B over the surface of the parietal pericardium PP, typically via a subxiphoid approach. After reaching the parietal pericardial surface, the access apparatus 10 is exposed and the sheath 11 can be withdrawn and removed. The tissue penetrating distal end 32 may be engaged against the parietal pericardium PP and embedded therein by rotating the tube 12 after the piercing members 36 have been positioned, as illustrated in FIG. 6B (see larger arrow). It will be appreciated that when the access tube 12 is rotated, the piercing members 36 should be relatively fixed to the access tube 12 or locked in place, so that they are sufficiently held in place when the access tube 12 is rotated. There are a variety of ways to accomplish holding the piercing members 36 in place when the access tube 12 is to be rotated.

As one example, each of the piercing members 36 is constructed to have a proper interference fit within its corresponding lumen 30. That is, each piercing member 36 is fitted into its respective lumen 30, such that the piercing member 36 does not rotate when the access tube 12 is rotated so that the tissue penetrating distal end 32 of the piercing member 36 can penetrate and engage the parietal pericardium PP, and such that the piercing member 36 may later be advanced into the pericardial space PS′ to form the supporting structure 38′. It will be appreciated that the materials and sizing used for constructing the interference fit can be accomplished by one of skill in the art, so long as the piercing members 36 do not rotate when the access tube 12 is to be rotated to engage the parietal pericardium PP and so long as it can be advanced into the pericardial space after engagement.

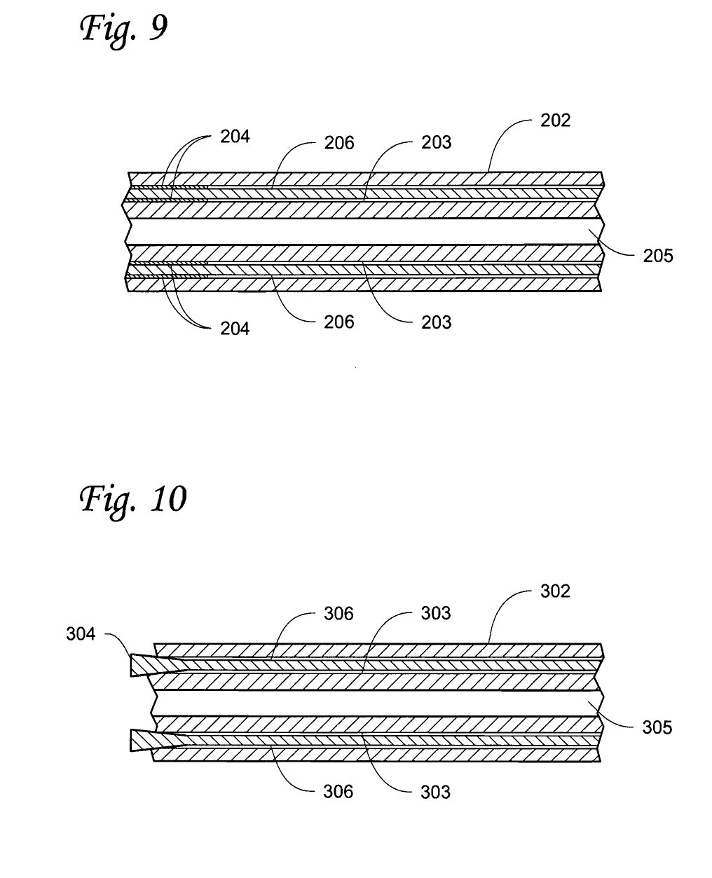

In another example, FIG. 9 shows an embodiment where each piercing member 206 may be held in place by an adhesive bond 204 disposed between each piercing member 206 and its corresponding lumen 203. See access tube 202 having central passage 205 in FIG. 9. The adhesive bond may be disposed substantially on a surface of at least one of each piercing member 206 or each lumen 203, and may be disposed along a length corresponding to the entire length of each lumen 203 or along a length corresponding to less than the entire length of each lumen 203. In one embodiment, the adhesive bond would be sufficiently strong over a certain rotational load, such that the piercing member 206 remains bonded to its respective lumen 203, when the access tube (i.e. 12) is rotated to engage the parietal pericardium PP. When the piercing member 206 has engaged the parietal pericardium PP and is ready to be advanced into the pericardial space PS, the adhesive bond can be broken between the piercing member 206 and lumen 203 by exerting a certain longitudinally applied force to allow the piercing member to advance and form the supporting structure (i.e. 38′). It will be appreciated that the force or load exerted by rotating the access tube in engaging the parietal pericardium is less than the force or load exerted by advancing the piercing member 206.

In still another embodiment, FIG. 10 shows an embodiment of a tapered extrusion 304 or cover which can be used to engage a proximal end of the piercing member 306, so that the piercing member 306 is maintained in a stationary position in its corresponding lumen 303. See access tube 302 having central passage 305 in FIG. 10. The tapered extrusion 304 can be passed over a proximal portion of the piercing member 306 and creates an interference fit between the lumen 303 and piercing member 306. The tapered extrusion 304 can be removed when the piercing member 306 has engaged the parietal pericardium PP and is ready to be advanced into the pericardial space PS to form the supporting structure (i.e. 38′). FIGS. 9 and 10 respectively illustrate an example of using an adhesive bond and a tapered extrusion.

The piercing members may be held in a variety of ways so that the access tube can be rotated to engage the parietal pericardium. It will be appreciated that the above examples may be modified as desired, suitable, and/or necessary. It further will be appreciated that the manner in which the piercing members are held is not limited to the drawings shown and that one of skill in the art can employ various configurations to achieve the necessary hold of the piercing members when the access tube is rotated to penetrate and engage an anatomic wall.

As yet another embodiment, it will be appreciated that the penetration may also be achieved by rotating each of the piercing members 36 (as shown by smaller arrow in FIG. 6B).

Turning again back to FIGS. 6A-6G, the tissue penetrating distal ends 32 engage the parietal pericardium PP, but do not engage or advance into the visceral pericardium VP or structures below. Thus, once engaged, the access tube 12 is able to draw the parietal pericardium PP away from the visceral pericardium VP to create an enlarged pericardial space (from PS to PS′).

In one embodiment, while the piercing member 36 is advanced forward in the lumen 30 and axially between the central passage 22 and an outside wall of the access tube 12, the distal portion 38 of the piercing member 36 that enters the pericardial space PS′ starts to form the supporting structure 38′. The piercing member 36 draws the parietal pericardium PP away from the visceral pericardium VP until the supporting structure 38′ has been at least partially formed so that the parietal pericardium PP can be released. As described above, it will be appreciated that when the apparatus is operated, the parietal pericardium may also be drawn away from the visceral pericardium until the piercing members 36 can form the supporting structures 38′.

It will be appreciated that all the piercing members 36 can be advanced into the pericardial space PS′, curled on themselves (at 60) to form the supporting structures 38′. The piercing members 36 can be advanced simultaneously or at separate times. When the piercing members 36 are advanced simultaneously, the anatomic wall, such as parietal pericardium PP is drawn away from the visceral pericardium, while the piercing members 36 form supporting structures 38′. That is, the nature of the material of the piercing members is such that, as soon as they have penetrated and engaged the parietal pericardium PP, the piercing members 36 can be advanced to start forming supporting structures 38′ (e.g., curling on themselves to form coils). Subsequently, larger dimensions of the supporting structure 38′ are continuously formed when the piercing members 36 are continuously advanced into the pericardial space PS′.

In another embodiment, at least one of the piercing members 36 remains holding the parietal pericardium PP and is not advanced into the pericardial space PS′ until the other piercing members have been advanced, either simultaneously or one at a time so as to form the supporting structure 38′.

In one example as shown in FIG. 6C, the piercing members 36 start to curl on themselves (at 60) to form the supporting structures 38 in the form of a coil 62. As the piercing member 36 is advanced into the pericardial space PS′, the piercing member 36 gradually expands the pericardial space PS′ without injuring either the parietal pericardium or the visceral pericardium, that is, the dimension of the supporting structure 38′ increases as the piercing member 36 is advanced, thereby expanding the pericardial space PS′ into a working space. When the piercing member 36 further continues to advance into the pericardial space PS′, the supporting structure 38′ is further enlarged. In one embodiment where the supporting structure 38′ is a coil 62, the dimension of the coil 62 is increased. As a result, the parietal pericardium PP can be tented and an enlarged working space is created, as shown in FIG. 6D. It will be appreciated that the amount that the piercing member(s) 36 are advanced may vary as necessary to create a suitable supporting structure(s) 38′ so that can provide a sufficient working space for any number of procedures to be performed.

After the working space is created, the access device 14 may be introduced through the access tube 12 and into the working space created, as shown in FIG. 6E. The access device 14 is then available to perform a wide variety of tasks and protocols. For example, it could be used for infusion or aspiration of fluids, drug delivery, diagnostic and therapeutic electrophysiology procedures, pacemaker lead implantation, defibrillator lead placement, transmysocardial revascularization, transmysocardial revascularization with drug delivery, placement of left ventricular assist devices, placement of arterial bypass graphs, in situ bypass, i.e., coronary artery-venous fistulae, placement of drug delivery depots, closure of the left atrial appendage, or the like.

In one embodiment as shown in FIG. 6F, the access device 14 can be used to introduce a guidewire 40 into the working space. Once the guidewire 40 is in place, the access device 14 may be withdrawn, for example, by being pulled back, leaving the guidewire passing through the access tube 12, as illustrated in FIG. 6G. The guidewire 40 may then be used to introduce a wide variety of catheters or other diagnostic or therapeutic devices in order to perform any of the procedures listed above. In an exemplary use, the guidewire 40 is used to introduce a catheter and related instruments for closing the left atrial appendage, as generally described U.S. Pat. No. 6,488,689.

Alternatively, the access device 114 may also be employed to introduce a guidewire, while also introducing contrast media.

In another embodiment as shown in FIG. 7, the supporting structures 38′ can be used to pull and draw the parietal pericardium PP away from the visceral pericardium VP to further enlarge and maintain the working space, as shown in FIG. 7. It will be appreciated that the supporting structures 38′ can be configured to constantly hold the parietal pericardium PP, for example, by the tightness of the coil, which would not relax under certain tension. That is, in the embodiment where each supporting structure 38′ is a coil, the natural propensity and the coiling of the supporting structure 38′ may be suitably strong to allow for a certain amount of pulling of the parietal pericardium PP away from the visceral pericardium VP before uncoiling would occur. The surface of the supporting structure 38′ has no edges that would injure the inner surface of the parietal pericardium PP or the visceral pericardium VP. In some examples, the surface of the supporting structure 38′ can be generally radiused or curved. In this embodiment, while the supporting structures 38′ are not in contact with the visceral pericardium VP, an expanded room is left for the access device 14 as well as the guidewire 40 to be operated in the working space.

It will be appreciated that the piercing members 36 are to be withdrawn from the working space when the intended operation in the enlarged working space is complete. In one embodiment, the piercing members 36 can be pulled from their proximal ends 34 at the proximal end 18 of the access tube 20, either as individual members or altogether at one time. By pulling from the proximal ends 18, the piercing members 36, such as the coils, will be straightened out as it is pulled back into lumen 30.

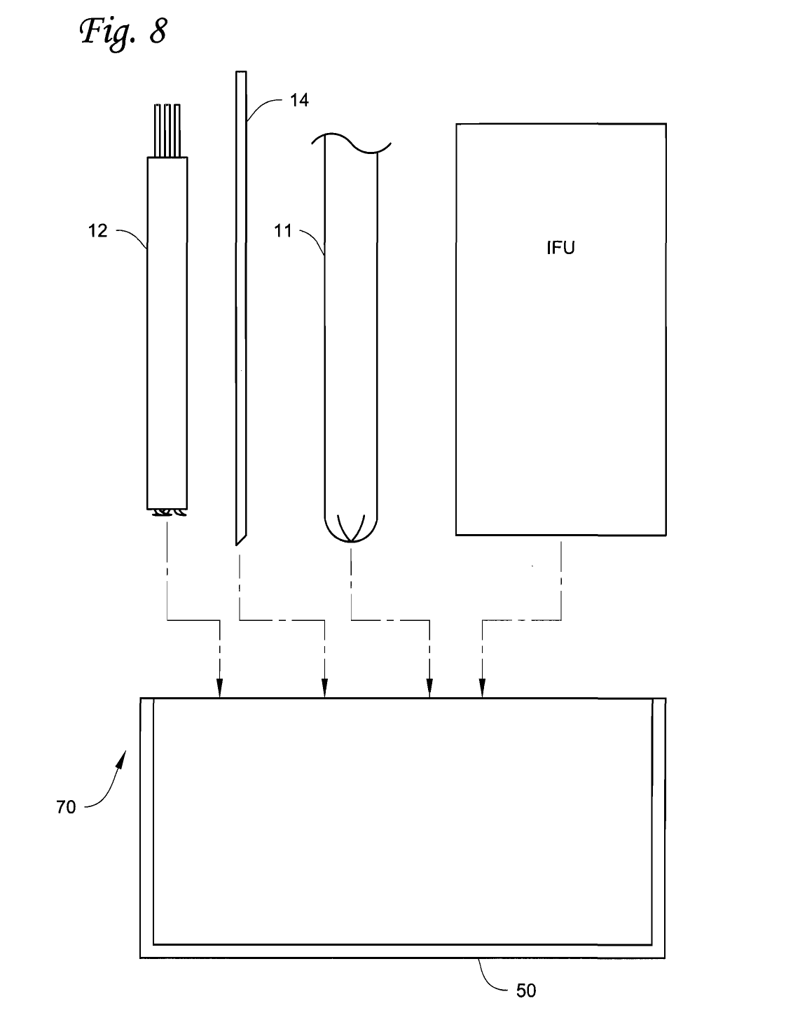

Referring now to FIG. 8, a kit 70 according to the present invention comprises at least an access tube 12 and instructions for use (IFU) setting forth a method according to the present disclosure for accessing an anatomic space. Optionally, the kit may further include an access device, such as access device 14, a sheath 11, as well as packaging 50, typically in the form of a box, pouch, tray, tube, or the like. The kit 70 could further include a guidewire and other components or instruments as known in the art and that may be useful for positioning the access tube and access device in performing the access methods. Instructions for use (IFU) can be printed on a separate sheet of paper in the form of a package insert, but could also be printed partly or wholly on the packaging itself. It will be appreciated that some of the apparatuses, such as the access tube 12, access device 14, etc, may be disposed after use.

While the above is a complete description of the preferred embodiments of the invention, various alternatives, modifications, and equivalents may be used. Therefore, the above description should not be taken as limiting the scope of the invention which is defined by the appended claims.

What is claimed is:

1. An apparatus for accessing an anatomic space having a wall with an outer surface and an inner surface, the apparatus comprising:
an elongated body having a central passage, an outside wall, and at least one lumen extending axially between the central passage and the outside wall of the elongated body, and
at least one piercing member carried by the lumen having a distal end configured to penetrate the wall, the piercing member having a distal portion and being configured to advance through the lumen and be exposed outside the elongated body, the piercing member being configured to form a supporting structure when exposed, and expand the anatomic space to create a working space inside the anatomic space without injuring the wall.

2. The apparatus as in claim 1, wherein the piercing member is configured such that as the piercing member is advanced, a dimension of the supporting structure increases.

3. The apparatus as in claim 1, wherein the piercing member is held within the lumen by one of an interference fit between the piercing member and the lumen, an adhesive bond between the piercing member and the lumen, and a tapered extrusion covering a proximate end of the piercing member and disposed between the piercing member and the lumen.

4. The apparatus as in claim 1, wherein a surface of the supporting structure is generally curved or radiused so that the supporting structure does not injure the inner surface of the anatomic wall.

5. The apparatus as in claim 1, further comprising a plurality of lumens extending axially between the central passage and the outside wall of the elongated body, each of which carries a piercing member therein.

6. A system for accessing an anatomic space having a wall with an outer surface, the system comprising:
an access tube having a central passage, an outside wall and at least one lumen extending axially between the central passage and the outside wall of the access tube, and at least one piercing member carried by the lumen, the piercing member having a distal end configured to penetrate the wall, and advance through the lumen and into the anatomic space, and configured to form a supporting structure and expand the anatomic space to create a working space inside the anatomic space without injuring the wall; and
an access device being configured to pass through the access tube and enter into the anatomic space after the working space has been created.

7. The system as in claim 6, wherein the access device comprises a needle and a guidewire configured to be positioned into the anatomic space through the needle.

8. The system as in claim 6, wherein the access device comprises a double needle structure, the needles being disposed directly adjacent each other and each needle including a proximate and distal end with a channel extending axially from the proximate end to the distal end, one of the needles is configured to introduce at least one of a guidewire and contrast media and the other needle is configured for imaging.

9. The system as in claim 6, further comprising a plurality of lumens extending axially between the central passage and the outside wall of the access tube, each of which carries a piercing member therein.

10. The system as in claim 6, further comprising a sheath configured to house the access tube, the sheath including a tapered distal end having a plurality of flexible portions configured to flex outward to provide an opening through which a distal end of the access tube is exposed when the sheath is retracted.

11. A kit for accessing a pericardial space between a visceral pericardium and a parietal pericardium, the kit comprising:
an access tube having a central passage, an outside wall, and at least one lumen extending axially between the central passage and the outside wall of the access tube, and at least one piercing member carried by the lumen, the piercing member having a distal end configured to penetrate the parietal pericardium, advance through the lumen and into the pericardial space, and configured to form a supporting structure and expand the pericardial space to create a working space inside the pericardial space without injuring the visceral pericardium and the parietal pericardium; and
a package for holding the access tube.

12. The kit as in claim 11, further comprising instructions for use whereby providing a method of using the access tube for accessing the anatomic space.

13. The kit as in claim 11, further comprising an access device being configured to pass through the access tube and enter into the pericardial space after the working space has been created.

14. The kit as in claim 11, further comprising a plurality of lumens extending axially between the central passage and the outside wall of the access tube, each of which carries a piercing member therein.

15. The kit as in claim 11, further comprising a sheath configured to house the access tube, the sheath including a tapered distal end having a plurality of flexible portions configured to flex outward to provide an opening through which a distal end of the access tube is exposed.

16. A method for accessing an anatomic space having a wall having an outer surface, the method comprising:
a) providing an access tube having a central passage, an outside wall, and at least one lumen extending axially between the central passage and the outside wall of the access tube, the lumen carrying therein a piercing member;
b) positioning a distal end of the piercing member proximate to the outer surface of the wall;
c) penetrating and engaging the distal end of the piercing member with the wall;
d) advancing the piercing member so that the distal end of the piercing member enters into the anatomic space; and
e) expanding the anatomic space to create a working space inside the anatomic space without injuring the anatomic wall by forming a supporting structure with the piercing member when the piercing member is further advanced after the distal end has entered the anatomic space.

17. The method as in claim 16, further comprising introducing an access device through the access tube, penetrating the wall and entering into the anatomic space while the supporting structure stabilizes the wall.

18. The method as in claim 16, further comprising pulling the piercing member to enlarge the working space.

19. The method as in claim 16, wherein a plurality of lumens extend axially between the central passage and the outside wall of the access tube, each of which carries a piercing member therein, and performing steps b)-e) with each additional piercing member.

20. The method as in claim 19, further comprising drawing the wall over the piercing member to raise the wall over the anatomic space until the supporting structure has been at least partially formed, and releasing drawing of the wall after the supporting structure has been partially formed.

21. The method as in claim 16, wherein the anatomic space is between a parietal pericardium and a visceral pericardium.

22. The method as in claim 16, wherein step c) further comprises rotating one of the access tube or the piercing member.
